(19) United States
(12) Patent Application Publication
(10) Pub. No.: US 2015/0051681 A1
(43) Pub. Date: Feb. 19, 2015

(54) METHODS AND SYSTEMS FOR ANODAL STIMULATION TO AFFECT CRANIAL AND OTHER NERVES

(71) Applicant: BOSTON SCIENTIFIC NEUROMODULATION CORPORATION, Valencia, CA (US)

(72) Inventor: Bradley Lawrence Hershey, Valencia, CA (US)

(21) Appl. No.: 14/462,248

(22) Filed: Aug. 18, 2014

Related U.S. Application Data
(60) Provisional application No. 61/867,485, filed on Aug. 19, 2013.

(51) Int. Cl. A61N 1/05

(52) U.S. Cl. CPC A61N 1/0551
USPC 607/117

(57) ABSTRACT

A method of stimulating patient nerve tissue includes implanting an electrical stimulation lead in a patient. The electrical stimulation lead includes multiple electrodes disposed thereon and the electrodes are implanted in an epidural space adjacent a spinal cord of the patient. The method further includes coupling the electrical stimulation lead to a control module; implanting the control module in the patient; and generating stimulation current in the control module and delivering the stimulation current to at least one of the electrodes on the electrical stimulation lead. That electrode is an anode and a cathode is provided on the control module. The method also includes electrically stimulating at least one patient nerve tissue using the stimulation current. The stimulated patient nerve tissue is disposed beyond the spinal cord and dorsal and ventral roots extending from the spinal cord.

### CROSS-REFERENCE TO RELATED APPLICATIONS

This application claims the benefit under 35 U.S.C. §119(e) of U.S. Provisional Patent Application Ser. No. 61/867,485, filed Aug. 19, 2013, which is incorporated herein by reference.

### FIELD

The present invention is directed to the area of implantable electrical stimulation systems and methods of making and using the systems. The present invention is also directed to implantable electrical stimulation leads employing anodal stimulation, as well as methods of making and using the leads and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

One embodiment is a method of stimulating patient nerve tissue. The method includes implanting an electrical stimulation lead in a patient. The electrical stimulation lead includes multiple electrodes disposed thereon and the electrodes are implanted in an epidural space adjacent a spinal cord of the patient. The method further includes coupling the electrical stimulation lead to a control module; implanting the control module in the patient; and generating stimulation current in the control module and delivering the stimulation current to at least one of the electrodes on the electrical stimulation lead. That electrode is an anode and a cathode is provided on the control module. The method also includes electrically stimulating at least one patient nerve tissue using the stimulation current. The stimulated patient nerve tissue is disposed beyond the spinal cord and dorsal and ventral roots extending from the spinal cord.

Another embodiment is a method of stimulating a cranial nerve of a patient. The method includes implanting an electrical stimulation lead in an epidural space adjacent a cervical region of a spinal cord of a patient. The electrical stimulation lead has multiple electrodes disposed thereon. The method further includes coupling the electrical stimulation lead to a control module; and generating stimulation current in the control module and delivering the stimulation current to one or more electrodes on the electrical stimulation lead. At least one of those electrodes is an anode. The method also includes electrically stimulating at least one cranial nerve of the patient using the stimulation current. That cranial nerve is disposed beyond the spinal cord and dorsal and ventral roots extending from the spinal cord.

Yet another embodiment is a method of treatment. The method includes implanting an electrical stimulation lead in an epidural space adjacent a cervical region of a spinal cord of a patient. The electrical stimulation lead has multiple electrodes disposed thereon. The method further includes coupling the electrical stimulation lead to a control module; and generating stimulation current in the control module and delivering the stimulation current to one or more electrodes on the electrical stimulation lead. At least one of those electrodes is an anode. The method also includes electrically stimulating patient tissue using the stimulation current to provide effective treatment of a disease, disorder, or dysfunction selected from frontal headache, epilepsy, trigeminal neuralgia, cluster headache, or migraine headache.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a paddle lead electrically coupled to a control module, according to the invention;

FIG. 2 is a schematic view of one embodiment of an electrical stimulation system that includes a percutaneous lead electrically coupled to a control module, according to the invention;

FIG. 3A is a schematic view of one embodiment of the control module of FIG. 1 configured and arranged to electrically couple to an elongated device, according to the invention;

FIG. 3B is a schematic view of one embodiment of a lead extension configured and arranged to electrically couple the elongated device of FIG. 2 to the control module of FIG. 1, according to the invention;

FIG. 4 is a schematic overview of one embodiment of components of a stimulation system, including an electronic subassembly disposed within a control module, according to the invention;

FIG. 5 is a schematic flowchart of one embodiment of a method of stimulating nerve tissue, according to the invention;

FIG. 6 is a schematic flowchart of one embodiment of a method of stimulating a cranial nerve, according to the invention; and

FIG. 7 is schematic flowchart of one embodiment of a method for treating a disease, disorder, or dysfunction using anodal stimulation, according to the invention.

### DETAILED DESCRIPTION

The present invention is directed to the area of implantable electrical stimulation systems and methods of making and using the systems. The present invention is also directed to implantable electrical stimulation leads employing anodal stimulation, as well as methods of making and using the leads and electrical stimulation systems.

Suitable implantable electrical stimulation systems include, but are not limited to, a least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,949,395; 7,244,150; 7,672,734; 7,761,165; 7,974,706; 8,175,710; 8,224,450; and 8,364,278; and U.S. Patent Application Publication No. 2007/0150036, all of which are incorporated by reference.

In conventional spinal cord stimulation, the lead is implanted in the epidural space of the spinal cord and cathodal electrical stimulation is provided with at least one of the electrodes on the lead acting as a cathode. The control module may be implanted, typically in the body cavity, and may include an anode or the anode may be distributed over one or more electrodes of the lead. Such cathodal electrical stimulation typically affects nerve tissue that is close to the stimulating electrode.

In contrast to cathodal stimulation, anodal stimulation can be effective to electrically stimulate nerves that are much further away from the stimulating electrode. Using one or more electrodes on the implanted lead as an anode, with the cathode disposed on the control module or distributed over two or more electrodes on the lead, anodal stimulation can be effective in stimulating nerve tissue beyond the spinal cord and its dorsal and ventral roots. Each anode sources current and each cathode sinks current.

As an example, a lead implanted within the epidural space of the cervical region (e.g., C4-C7) of the spinal cord is typically useful, when employing cathodal stimulation, for addressing pain or other dysfunctions of the upper extremities (e.g., arms, hands, and the like). The upper extremities include nerves coupled to the roots of the spinal cord in the cervical region (e.g., C4-C7).

In contrast, anodal stimulation from a lead implanted with in the epidural space of the cervical region (e.g., C4-C7 or C5-C7) of the spinal cord can be used to stimulate cranial nerve tissue including nerve tissue in the face, jaw, or back of the head. This can occur even though the cranial nerve tissue is beyond the spinal cord and the roots of the spinal cord in the cervical region and even though such nerve tissue does not connect to the roots extending from the spinal cord in the C4-C7 or C5-C7 cervical region. Cranial nerve tissue that can be stimulated can include, for example, an occipital nerve or a trigeminal nerve. Such stimulation could be used to treat diseases, disorder, or dysfunctions such as, for example, frontal headache, epilepsy, trigeminal neuralgia, cluster headache, and migraine. Thus, these diseases, disorders, or dysfunctions can be treated using an electrical stimulation lead implanted in the epidural space in the cervical region (e.g., C4-C7 or C5-C7) of the spinal cord. Such treatment can include, for example, pain reduction; reduction in the severity of one or more symptoms associated with the disease, disorder, or dysfunction; reduction in the frequency of one or more symptoms associated with the disease, disorder, or dysfunction; reduction in the duration of one or more symptoms associated with the disease, disorder, or dysfunction; and the like.

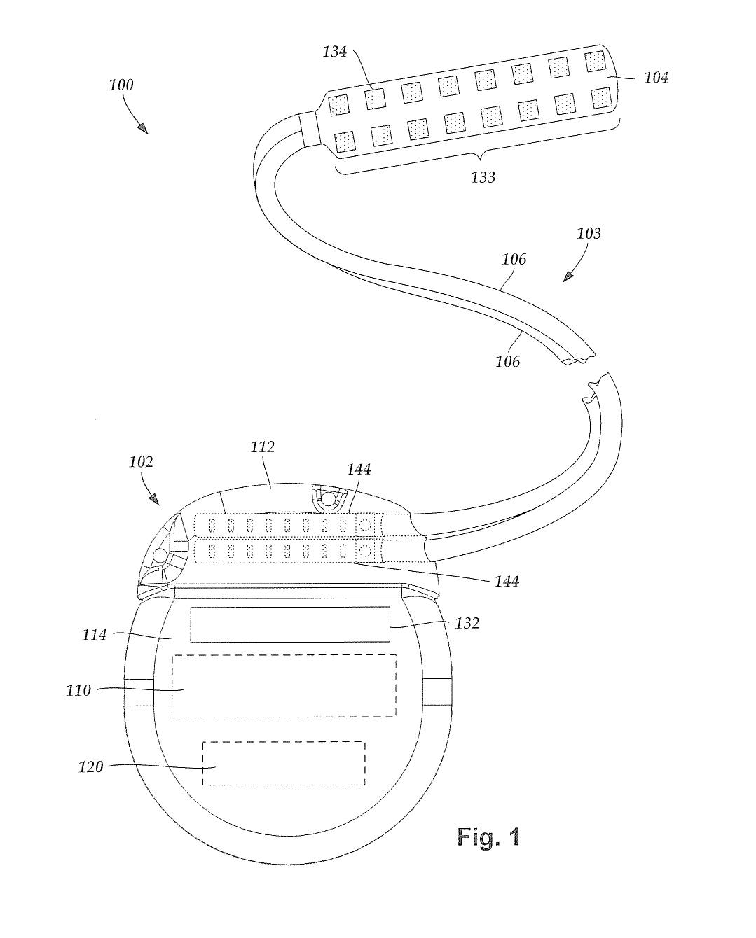

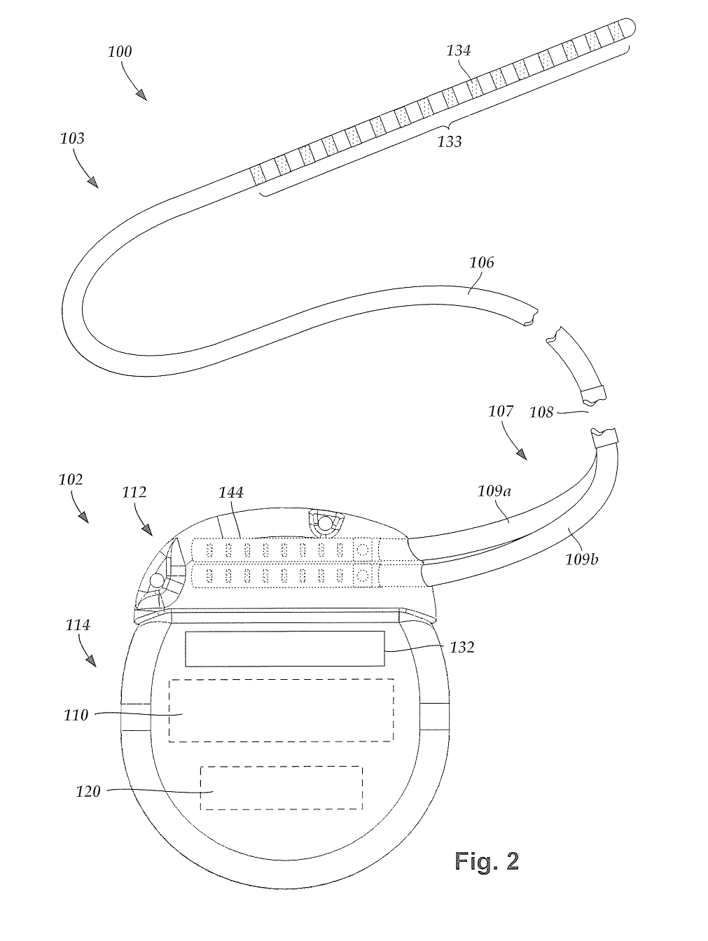

FIG. 1 illustrates schematically one embodiment of an electrical stimulation system 100. The electrical stimulation system includes a control module (e.g., a stimulator or pulse generator) 102 and a lead 103 coupleable to the control module 102. The lead 103 includes a paddle body 104 and one or more lead bodies 106. In FIG. 1, the lead 103 is shown having two lead bodies 106. It will be understood that the lead 103 can include any suitable number of lead bodies including, for example, one, two, three, four, five, six, seven, eight or more lead bodies 106. An array of electrodes 133, such as electrode 134, is disposed on the paddle body 104, and an array of terminals (e.g., 210 in FIG. 2A-2B) is disposed along each of the one or more lead bodies 106.

In at least some embodiments, the control module 102 also includes one or more electrodes 132 disposed thereon. The electrode(s) 132 can be individual and discrete or may include a substantial portion of the control module 102 such as the electronics housing 114 (discussed below).

It will be understood that the electrical stimulation system can include more, fewer, or different components and can have a variety of different configurations including those configurations disclosed in the electrical stimulation system references cited herein. For example, instead of a paddle body, the electrodes can be disposed in an array at or near the distal end of a lead body forming a percutaneous lead.

FIG. 2 illustrates schematically another embodiment of the electrical stimulation system 100, where the lead 103 is a percutaneous lead. In FIG. 2, the electrodes 134 are shown disposed along the one or more lead bodies 106. In at least some embodiments, the lead 103 is isodiametric along a longitudinal length of the lead body 106.

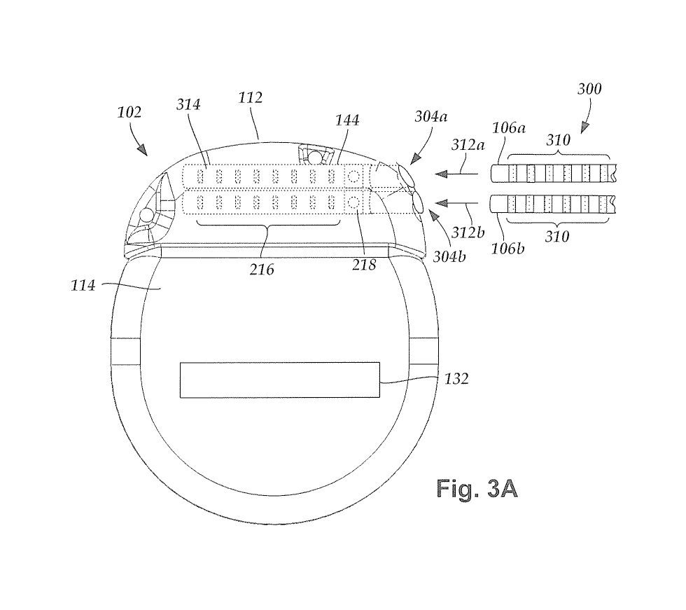

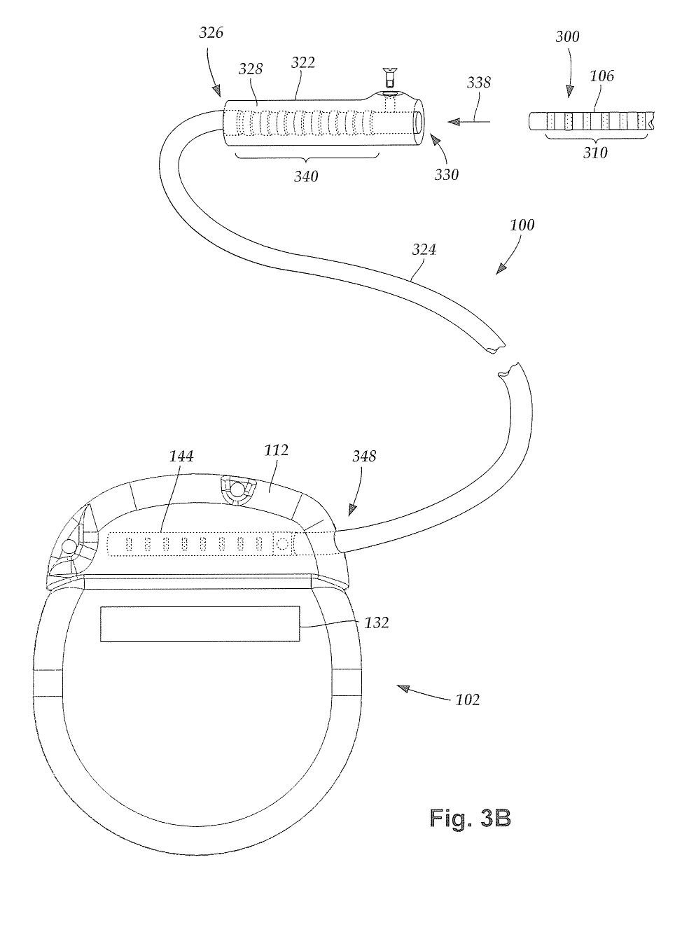

The lead 103 can be coupled to the control module 102 in any suitable manner. In FIG. 1, the lead 103 is shown coupling directly to the control module 102. In at least some other embodiments, the lead 103 couples to the control module 102 via one or more intermediate devices (300 in FIGS. 3A-3B). For example, in at least some embodiments one or more lead extensions 324 (see e.g., FIG. 3B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

In FIG. 2, the electrical stimulation system 100 is shown having a splitter 207 configured and arranged for facilitating coupling of the lead 103 to the control module 102. The splitter 207 includes a splitter connector 208 configured to couple to a proximal end of the lead 103, and one or more splitter tails 209a and 209b configured and arranged to couple to the control module 102 (or another splitter, a lead extension, an adaptor, or the like).

The control module 102 typically includes a connector housing 112 and a sealed electronics housing 114. An electronic subassembly 110 and an optional power source 120 are disposed in the electronics housing 114. A control module connector 144 is disposed in the connector housing 112. The control module connector 144 is configured and arranged to make an electrical connection between the lead 103 and the electronic subassembly 110 of the control module 102. The optional electrode(s) 132 disposed on the control module are electrically coupled to the electronic subassembly 110 of the control module 10.

The electrical stimulation system or components of the electrical stimulation system, including the paddle body 104, the one or more of the lead bodies 106, and the control module 102, are typically implanted into the body of a patient. The electrical stimulation system can be used for a variety of applications including, but not limited to neural stimulation, spinal cord stimulation, muscle stimulation, and the like.

The lead electrodes 134 and optional control module electrode(s) 132 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium.

Any suitable number of electrodes 134 can be disposed on the lead including, for example, four, five, six, seven, eight, nine, ten, eleven, twelve, fourteen, sixteen, twenty-four, thirty-two, or more electrodes 134. In the case of paddle leads, the electrodes 134 can be disposed on the paddle body 104 in any suitable arrangement. In FIG. 1, the electrodes 134 are arranged into two columns, where each column has eight electrodes 134.

The electrodes of the paddle body 104 (or one or more lead bodies 106) are typically disposed in, or separated by, a non-conductive, biocompatible material such as, for example, silicone, polyurethane, polyetheretherketone (“PEEK”), epoxy, and the like or combinations thereof. The one or more lead bodies 106 and, if applicable, the paddle body 104 may be formed in the desired shape by any process including, for example, molding (including injection molding), casting, and the like. The non-conductive material typically extends from the distal ends of the one or more lead bodies 106 to the proximal end of each of the one or more lead bodies 106.

In the case of paddle leads, the non-conductive material typically extends from the paddle body 104 to the proximal end of each of the one or more lead bodies 106. Additionally, the non-conductive, biocompatible material of the paddle body 104 and the one or more lead bodies 106 may be the same or different. Moreover, the paddle body 104 and the one or more lead bodies 106 may be a unitary structure or can be formed as two separate structures that are permanently or detachably coupled together.

Terminals (e.g., 310 in FIGS. 3A-3B) are typically disposed along the proximal end of the one or more lead bodies 106 of the electrical stimulation system 100 (as well as any splitters, lead extensions, adaptors, or the like) for electrical connection to corresponding connector contacts (e.g., 314 in FIGS. 3A-3B). The connector contacts are disposed in connectors (e.g., 144 in FIGS. 1-3B; and 322 FIG. 3B) which, in turn, are disposed on, for example, the control module 102 (or a lead extension, a splitter, an adaptor, or the like). Electrically conductive wires, cables, or the like (not shown) extend from the terminals to the electrodes 134. Typically, one or more electrodes 134 are electrically coupled to each terminal. In at least some embodiments, each terminal is only connected to one electrode 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the one or more lead bodies 106, for example, for inserting a stylet to facilitate placement of the one or more lead bodies 106 within a body of a patient. Additionally, there may be one or more lumens (not shown) that open at, or near, the distal end of the one or more lead bodies 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably sealable at the distal end.

FIG. 3A is a schematic side view of one embodiment of a proximal end of one or more elongated devices 300 configured and arranged for coupling to one embodiment of the control module connector 144. The one or more elongated devices may include, for example, one or more of the lead bodies 106 of FIG. 1, one or more intermediate devices (e.g., a splitter, the lead extension 324 of FIG. 3B, an adaptor, or the like or combinations thereof), or a combination thereof.

The control module connector 144 defines at least one port into which a proximal end of the elongated device 300 can be inserted, as shown by directional arrows 312a and 312b. In FIG. 3A (and in other figures), the connector housing 112 is shown having two ports 304a and 304b. The connector housing 112 can define any suitable number of ports including, for example, one, two, three, four, five, six, seven, eight, or more ports.

The control module connector 144 also includes a plurality of connector contacts, such as connector contact 314, disposed within each port 304a and 304b. When the elongated device 300 is inserted into the ports 304a and 304b, the connector contacts 314 can be aligned with a plurality of terminals 310 disposed along the proximal end(s) of the elongated device(s) 300 to electrically couple the control module 102 to the electrodes (134 of FIG. 1) disposed on the paddle body 104 of the lead 103. Examples of connectors in control modules are found in, for example, U.S. Pat. Nos. 7,244,150 and 8,224,450, which are incorporated by reference.

FIG. 3B is a schematic side view of another embodiment of the electrical stimulation system 100. The electrical stimulation system 100 includes a lead extension 324 that is configured and arranged to couple one or more elongated devices 300 (e.g., one of the lead bodies 106 of FIGS. 1 and 2, the splitter 207 of FIG. 2, an adaptor, another lead extension, or the like or combinations thereof) to the control module 102. In FIG. 3B, the lead extension 324 is shown coupled to a single port 304 defined in the control module connector 144. Additionally, the lead extension 324 is shown configured and arranged to couple to a single elongated device 300. In alternate embodiments, the lead extension 324 is configured and arranged to couple to multiple ports 304 defined in the control module connector 144, or to receive multiple elongated devices 300, or both.

A lead extension connector 322 is disposed on the lead extension 324. In FIG. 3B, the lead extension connector 322 is shown disposed at a distal end 326 of the lead extension 324. The lead extension connector 322 includes a connector housing 328. The connector housing 328 defines at least one port 330 into which terminals 310 of the elongated device 300 can be inserted, as shown by directional arrow 338. The connector housing 328 also includes a plurality of connector contacts, such as connector contact 340. When the elongated device 300 is inserted into the port 330, the connector contacts 240 disposed in the connector housing 328 can be aligned with the terminals 310 of the elongated device 300 to electrically couple the lead extension 324 to the electrodes (134 of FIGS. 1 and 2) disposed along the lead (103 in FIGS. 1 and 2).

In at least some embodiments, the proximal end of the lead extension 324 is similarly configured and arranged as a proximal end of the lead 103 (or other elongated device 300). The lead extension 324 may include a plurality of electrically conductive wires (not shown) that electrically couple the connector contacts 340 to a proximal end 348 of the lead extension 324 that is opposite to the distal end 326. In at least some embodiments, the conductive wires disposed in the lead extension 324 can be electrically coupled to a plurality of terminals (not shown) disposed along the proximal end 348 of the lead extension 324. In at least some embodiments, the proximal end 348 of the lead extension 324 is configured and arranged for insertion into a connector disposed in another lead extension (or another intermediate device). In other embodiments (and as shown in FIG. 3B), the proximal end 348 of the lead extension 324 is configured and arranged for insertion into the control module connector 144.

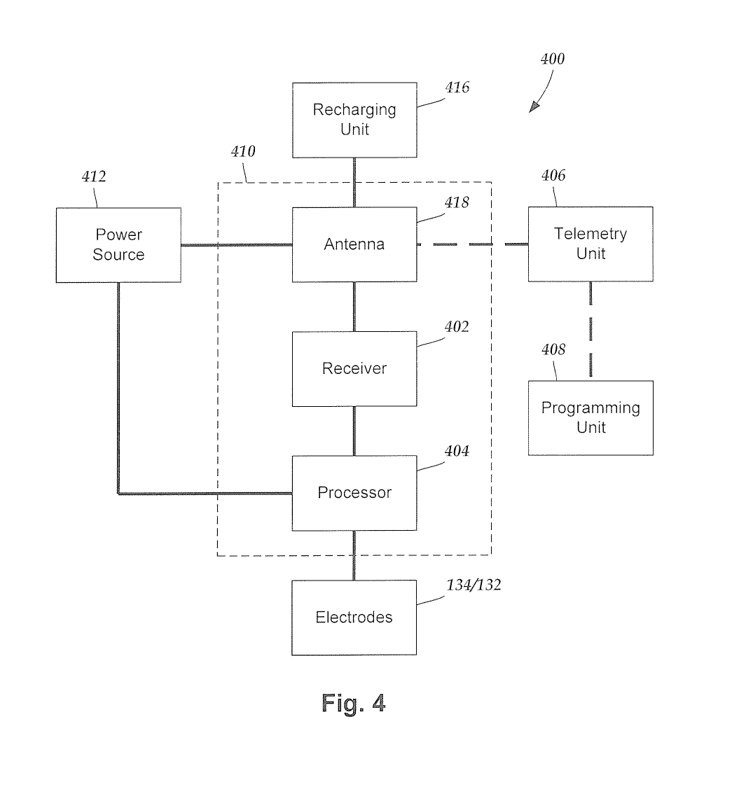

FIG. 4 is a schematic overview of one embodiment of components of an electrical stimulation system 400 including an electronic subassembly 410 disposed within a control module. It will be understood that the electrical stimulation system can include more, fewer, or different components and can have a variety of different configurations including those configurations disclosed in the stimulator references cited herein.

Some of the components (for example, a power source 412, an antenna 418, a receiver 402, and a processor 404) of the electrical stimulation system can be positioned on one or more circuit boards or similar carriers within a sealed housing of an implantable pulse generator, if desired. Any power source 412 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 418 or a secondary antenna. The external power source can be in a device that is mounted on the skin of the user or in a unit that is provided near the user on a permanent or periodic basis.

If the power source 412 is a rechargeable battery, the battery may be recharged using the optional antenna 418, if desired. Power can be provided to the battery for recharging by inductively coupling the battery through the antenna to a recharging unit 416 external to the user. Examples of such arrangements can be found in the references identified above.

In one embodiment, electrical current is emitted by the electrodes 134 on the paddle or lead body (or electrode(s) 132 on the control module) to stimulate nerve fibers, muscle fibers, or other body tissues near the electrical stimulation system. The processor 404 is generally included to control the timing and electrical characteristics of the electrical stimulation system. For example, the processor 404 can, if desired, control one or more of the timing, frequency, strength, duration, and waveform of the pulses. In addition, the processor 404 can select which electrodes can be used to provide stimulation, if desired. In some embodiments, the processor 404 selects which electrode(s) are cathodes and which electrode(s) are anodes. In some embodiments, the processor 404 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 408 that, for example, allows modification of pulse characteristics. In the illustrated embodiment, the processor 404 is coupled to a receiver 402 which, in turn, is coupled to the optional antenna 418. This allows the processor 404 to receive instructions from an external source to, for example, direct the pulse characteristics and the selection of electrodes, if desired.

In one embodiment, the antenna 418 is capable of receiving signals (e.g., RF signals) from an external telemetry unit 406 which is programmed by the programming unit 408. The programming unit 408 can be external to, or part of, the telemetry unit 406. The telemetry unit 406 can be a device that is worn on the skin of the user or can be carried by the user and can have a form similar to a pager, cellular phone, or remote control, if desired. As another alternative, the telemetry unit 406 may not be worn or carried by the user but may only be available at a home station or at a clinician's office. The programming unit 408 can be any unit that can provide information to the telemetry unit 406 for transmission to the electrical stimulation system 400. The programming unit 408 can be part of the telemetry unit 406 or can provide signals or information to the telemetry unit 406 via a wireless or wired connection. One example of a suitable programming unit is a computer operated by the user or clinician to send signals to the telemetry unit 406.

The signals sent to the processor 404 via the antenna 418 and the receiver 402 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 400 to cease operation, to start operation, to start charging the battery, or to stop charging the battery. In other embodiments, the stimulation system does not include the antenna 418 or receiver 402 and the processor 404 operates as programmed.

Optionally, the electrical stimulation system 400 may include a transmitter (not shown) coupled to the processor 404 and the antenna 418 for transmitting signals back to the telemetry unit 406 or another unit capable of receiving the signals. For example, the electrical stimulation system 400 may transmit signals indicating whether the electrical stimulation system 400 is operating properly or not or indicating when the battery needs to be charged or the level of charge remaining in the battery. The processor 404 may also be capable of transmitting information about the pulse characteristics so that a user or clinician can determine or verify the characteristics.

In operation, one or more of the electrodes of the lead are selected to act as an anode. The anode(s) source current flows through tissue to the selected nerve tissue to be stimulated. The current flows to the cathode(s) which sink current. The cathode(s) can include an electrode on the housing of the control module or one or more electrodes of the lead or any combination thereof. If the cathode(s) include a lead electrode, it is preferable that more than one lead electrode be selected to disperse or dilute the cathodal current over a wider physical range.

The selection of which electrodes act as anode(s) and cathode(s) permits a practitioner to guide the stimulation by steering the current to the desired stimulation site. In some embodiments, the selection of anode(s) and cathode(s) may vary over time at regular or irregular intervals and according to regular or irregular patterns.

Electrical stimulation parameters for effective treatment can vary depending on a number of factors including, but not limited to, the size of the electrodes, the placement of the lead, the target tissue to be stimulated, the patient's body size, shape, and configuration, and the like. Examples of stimulation parameters include a stimulation current in the range of 0.05 to 100 milliamps, stimulation pulse width in the range of 50 to 1000 microseconds, and a pulse frequency in the range of 1 to 1000 Hertz. A practitioner, however, will often select the appropriate stimulation parameters based on experience and experimentation.

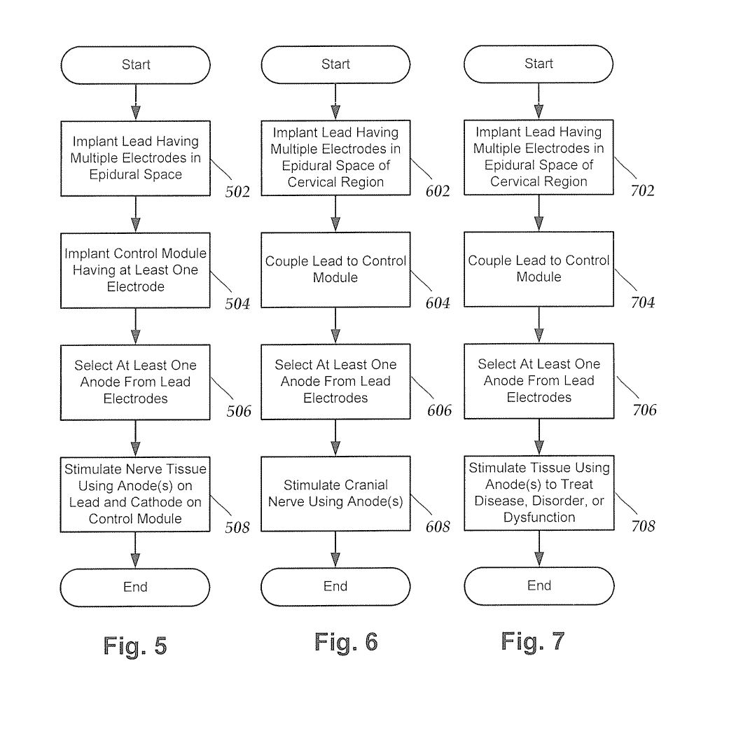

FIG. 5 illustrates one embodiment of a method of stimulating patient nerve tissue. The method includes implanting an electrical stimulation lead, such as any one of the leads illustrated in FIGS. 1-3B, in the epidural space of a patient (step 502). As describe above, the electrical stimulation lead includes multiple electrodes disposed thereon. The electrical stimulation lead is coupled to a control module, such as any one of the control modules illustrated in FIGS. 1-3B, that is also implanted in the patient (step 504). At least one of the lead electrodes is selected to be an anode (step 506). Stimulation current is generated in the control module and delivered to the anode(s) (step 508). A cathode is provided on the control module. This results in electrically stimulating patient nerve tissue using the stimulation current to provide anodal stimulation. In at least some embodiments, the patient nerve tissue is disposed beyond the spinal cord and dorsal and ventral roots extending from the spinal cord. For example, if the lead is implanted into the epidural space of the cervical region (e.g., C4-C7 or C5-C7) of the spinal cord, the stimulated nerve tissue may include a cranial nerve, such as an occipital nerve or a trigeminal nerve.

FIG. 6 illustrates an embodiment of a method of stimulating a cranial nerve of a patient. The method includes implanting an electrical stimulation lead, such as any one of the leads illustrated in FIGS. 1-3B, in an epidural space adjacent a cervical region of a spinal cord of a patient (step 602). The electrical stimulation lead has multiple electrodes disposed thereon. The electrical stimulation lead is coupled to a control module, such as any one of the control modules illustrated in FIGS. 1-3B, (step 604). At least one of the electrodes on the lead is selected as an anode and stimulation current is generated in the control module and delivered to one or more electrodes on the electrical stimulation lead (step 606). The corresponding cathode can be one or more electrodes on the control module or on the lead or any combination thereof. The stimulation current is used to electrically stimulate at least one cranial nerve of the patient (step 608). That cranial nerve is disposed beyond the spinal cord and dorsal and ventral roots extending from the spinal cord. The stimulated nerve can be, for example, an occipital nerve or a trigeminal nerve.

FIG. 6 illustrates an embodiment of a method of treatment. The method includes implanting an electrical stimulation lead, such as any one of the leads illustrated in FIGS. 1-3B, in an epidural space adjacent a cervical region of a spinal cord of a patient (step 702). The electrical stimulation lead has multiple electrodes disposed thereon. The electrical stimulation lead is coupled to a control module, such as any one of the control modules illustrated in FIGS. 1-3B, (step 704). At least one of the electrodes on the lead is selected as an anode and stimulation current is generated in the control module and delivered to one or more electrodes on the electrical stimulation lead (step 706). The corresponding cathode can be one or more electrodes on the control module or on the lead or any combination thereof. The stimulation current is used to stimulate tissue to provide effective treatment of a disease, disorder, or dysfunction selected from frontal headache, epilepsy, trigeminal neuralgia, cluster headache, or migraine headache (step 708). For example, the lead can be implanted into the epidural space of the cervical region (e.g., C4-C7 or C5-C7) of the spinal cord to stimulate a cranial nerve, such as an occipital nerve or a trigeminal nerve, to treat the disease, disorder, or dysfunction.

It will be recognized that in any of the methods described above, the electrode(s) selected to be anode(s) and cathode(s) can be changed over time according to any regular or irregular pattern and at any regular or irregular period of time. It will also be recognized that the stimulation current can be provided in a continuous or pulsed arrangement.

It will be understood that each block of the flowchart illustrations, and any combination of blocks in the flowchart illustrations, as well any portion of the control module, systems, and methods disclosed herein, can be implemented by computer program instructions. These program instructions may be provided to a processor to produce a machine, such that the instructions, which execute on the processor, create means for implementing the actions specified in the flowchart block or blocks or described for the sensor, imager, control module, systems and methods disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

The above specification provides a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. A method of stimulating patient nerve tissue, the method comprising:
implanting an electrical stimulation lead in a patient, the electrical stimulation lead comprising a plurality of electrodes disposed thereon, wherein the plurality of electrodes are implanted in an epidural space adjacent a spinal cord of the patient;
coupling the electrical stimulation lead to a control module;
implanting the control module in the patient;
generating stimulation current in the control module and delivering the stimulation current to at least one electrode of the plurality of electrodes on the electrical stimulation lead, wherein the at least one electrode is an anode and a cathode is provided on the control module; and
electrically stimulating at least one patient nerve tissue using the stimulation current, wherein the patient nerve tissue is disposed beyond the spinal cord and dorsal and ventral roots extending from the spinal cord.

2. The method of claim 1, wherein implanting the electrical stimulation lead comprises implanting the plurality of electrodes in the epidural space of a cervical region of the spinal cord.

3. The method of claim 2, wherein implanting the electrical stimulation lead comprises implanting the plurality of electrodes in the epidural space of the C5-C7 cervical region of the spinal cord.

4. The method of claim 2, wherein electrically stimulating at least one patient nerve tissue comprises electrically stimulating a cranial nerve of the patient.

5. The method of claim 4, wherein the cranial nerve is an occipital nerve.

6. The method of claim 4, wherein the cranial nerve is a trigeminal nerve.

7. The method of claim 4, wherein electrically stimulating at least one patient nerve tissue comprises electrically stimulating the at least one patient nerve tissue to treat a disease or dysfunction selected from frontal headache, epilepsy, trigeminal neuralgia, cluster headache, or migraine headache.

8. A method of stimulating a cranial nerve of a patient, the method comprising:
implanting an electrical stimulation lead in an epidural space adjacent a cervical region of a spinal cord of a patient, the electrical stimulation lead having a plurality of electrodes disposed thereon;
coupling the electrical stimulation lead to a control module;
generating stimulation current in the control module and delivering the stimulation current to one or more electrodes on the electrical stimulation lead, wherein at least one of the one or more electrodes is an anode; and
electrically stimulating at least one cranial nerve of the patient using the stimulation current, wherein the at least one cranial nerve is disposed beyond the spinal cord and dorsal and ventral roots extending from the spinal cord.

9. The method of claim 8, wherein implanting the electrical stimulation lead comprises implanting the plurality of electrodes in the epidural space of the C5-C7 cervical region of the spinal cord.

10. The method of claim 8, wherein the cranial nerve is an occipital nerve.

11. The method of claim 8, wherein the cranial nerve is a trigeminal nerve.

12. The method of claim 8, further comprising implanting the control module.

13. The method of claim 12, wherein generating stimulation current and delivering the stimulation current comprises generating the stimulation current and delivering the stimulation current using a cathode disposed on the control module.

14. The method of claim 8, wherein generating stimulation current and delivering the stimulation current comprises generating stimulation current and delivering the stimulation current using at least two of the electrodes on the stimulation lead as cathodes.

15. A method of treatment, the method comprising:
implanting an electrical stimulation lead in an epidural space adjacent a cervical region of a spinal cord of a patient, the electrical stimulation lead having a plurality of electrodes disposed thereon;
coupling the electrical stimulation lead to a control module;
generating stimulation current in the control module and delivering the stimulation current to one or more electrodes on the electrical stimulation lead, wherein at least one of the one or more electrodes is an anode; and
electrically stimulating patient tissue using the stimulation current to provide effective treatment of a disease, disorder, or dysfunction selected from frontal headache, epilepsy, trigeminal neuralgia, cluster headache, or migraine headache.

16. The method of claim 15, wherein implanting the electrical stimulation lead comprises implanting the plurality of electrodes in the epidural space of the C5-C7 cervical region of the spinal cord.

17. The method of claim 15, wherein electrically stimulating patient tissue comprises electrically stimulating a cranial nerve of the patient.

18. The method of claim 17, wherein the cranial nerve is an occipital nerve.

19. The method of claim 17, wherein the cranial nerve is a trigeminal nerve.

20. The method of claim 15, further comprising implanting the control module, wherein generating stimulation current and delivering the stimulation current comprises generating the stimulation current and delivering the stimulation current using a cathode disposed on the control module.
